(19) United States
(12) Patent Application Publication
(10) Pub. No.: US 2011/0207689 A1
(43) Pub. Date: Aug. 25, 2011

(54) THE TREATMENT OF HEARING LOSS

(75) Inventors: Srdjan Vlajkovic, Auckland (NZ); Peter Rowland Thorne, Auckland (NZ); Gary David Housley, New South Wales (AU)

(73) Assignee: Auckland UniServices Limited

(21) Appl. No.: 13/058,437

(22) PCT Filed: Aug. 11, 2009
(86) PCT No.: PCT/NZ09/00164

§ 371 Date: May 6, 2011

(30) Foreign Application Priority Data
Aug. 11, 2008 (NZ) 570469

(51) Int. Cl. A61K 31/7076; A61P 27/16

(52) U.S. Cl. 514/46

(57) ABSTRACT

The invention provides a method of treating noise-induced hearing loss, the method including the step of administering an A₁ adenosine receptor agonist to a patient in need thereof. In a particularly preferred embodiment the A₁ adenosine receptor agonist is a selective A₁ adenosine receptor agonist.

### FIELD OF THE INVENTION

The invention in general terms relates to a method of treating noise-induced hearing loss by administering an A₁ adenosine receptor agonist to a patient in need thereof.

### BACKGROUND

Hearing impairment is a significant health and social problem. One of the most common causes of hearing loss is excessive exposure to noise. This problem is particularly common in the military and in industrial settings (construction workers, mining, forestry and airline industry) where conventional hearing conservation programmes are difficult to operate. Some leisure activities (shooting, listening to loud music) may also lead to accidental hearing loss. USA health statistics indicate that hearing loss affects more than 25 million Americans at a cost of 50 billion dollars each year, which is more than the combined financial impact of multiple sclerosis, stroke, epilepsy, spinal injury, Huntington's and Parkinson's disease [1]. An estimated 10-13% of the New Zealand population is affected by significant hearing loss, and about one third owe the hearing loss to damage caused by excessive noise.

Noise-induced hearing loss can be caused by a one-time exposure to loud sound, as well as by repeated exposure to noise over an extended period of time. Standards set by Occupational Safety and Health (OSH) in New Zealand indicate that continued exposure to noise over 85 dB will eventually harm hearing.

Exposure to impulse or continuous noise may cause permanent or temporary hearing loss. The term ‘temporary threshold shift’ (TTS) is used to indicate a transient impairment of auditory function due to noise trauma, which usually disappears within about one week after exposure to loud noise. ‘Permanent threshold shift’ (PTS) occurs when post-exposure hearing thresholds have stabilised at reduced levels.

The majority of the hearing loss arises from injury to the sensory system of the inner ear. Whilst treatments exist for middle ear conditions, there are virtually no treatments that can ameliorate the damage to the inner ear pathology and reduce the impact of sensorineural hearing loss. There is increasing evidence that oxidative stress and the production of reactive oxygen species (ROS) are key elements in the pathogenesis of many forms of cochlear injury, for example from noise exposure, cytotoxic drugs and aging. Oxidative stress, along with neurotoxicity of glutamate, is being viewed almost as a unifying mechanism underlying most cochlear damage and hearing loss [2,3]. Thus compounds that target mechanisms underlying oxidative stress offer considerable potential as therapies for hearing loss. Adenosine receptor agonists have been successfully used in the treatment of ischemic brain and cardiac injury and are proving to have extraordinary cytoprotective functions. Adenosine receptors have been identified in the cochlea and adenosine levels are known to rise in cochlear fluids with noise exposure [4,5].

The use of the adenosine signalling system is known to have relevance to hearing. Animal studies have demonstrated that adenosine agonists can be useful prophylactically to prevent acquired hearing loss [6-9]. Pre-treatment with the non-selective A₁ adenosine receptor agonist R—N6-phenylisopropyladenosine (R-PIA) showed better preservation of auditory thresholds in the noise-exposed cochlea and increased survival of the outer hair cells as a result of prophylactic use [6]. R-PIA, however, was not applied after noise exposure and its effect on cochlear recovery from noise exposure is unknown. Moreover, R-PIA is not a selective adenosine receptor agonist, and it activates adenosine receptors which may have opposite effects on cochlear function, e.g. A₁ and A_{2A} receptors.

Clearly, instances of exposure to excessive noise may not always be predicted and thus prophylactic options are of limited use. If exposure to excessive noise is predictable then preventative options can be taken, such as use of ear plugs for example. Accordingly, it is essential to develop therapies for noise-induced hearing loss that can ameliorate injury to delicate structures of the inner ear and reduce hearing loss that result from exposure to excessive noise. Pharmacological therapies are currently not available for noise-induced hearing loss treatment, and hearing aids and cochlear implants are the only possibility offered to patients suffering from this condition.

The animal studies with prophylactic R-PIA have employed topical delivery to the round window membrane (RWM) of the cochlea due to systemic (cardiovascular) side effects. Whilst topical delivery of compounds to the RWM is commonly used in clinical practice, it is a surgical procedure and has some other disadvantages. Even though the RWM is the most surgically accessible route for drug delivery to the inner ear substances placed on the RWM do not distribute evenly through the cochlea [10]. Systemic drug administration (oral, parenteral) is preferable in clinical practice, as it eliminates the risk of a surgical procedure required to deliver drugs onto the RWM.

### OBJECT OF THE INVENTION

It is an object of the invention to provide a treatment for hearing loss that overcomes at least one of the disadvantages of the prior art or at least to provide the public with a useful choice.

### SUMMARY OF THE INVENTION

The invention in a first aspect provides a method of treating noise-induced hearing loss, the method including the step of administering an A₁ adenosine receptor agonist.

The invention in a second aspect provides a method of treating tissue injury to the cochlea after noise exposure, the method including the step of administering an A₁ adenosine receptor agonist.

Preferably the A₁ adenosine receptor agonist is a selective A₁ adenosine receptor agonist.

Preferably the selective A₁ adenosine receptor agonist is selected from the group including N6-cyclopentyl adenosine (CPA), 2-Chloro-N⁶-cyclopentyl adenosine (CCPA), S—N⁶-(2-endo-norbornyl)adenosine [S(−)-ENBA], adenosine amine congener (ADAC), ([1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide) (AMP579), N—[R-(2-Benzothiazolyl)thio-2-propyl]-2-chloroadenosine (NNC-21-0136), N-[(1S, trans)-2-hydroxycyclopentyl]adenosine (GR79236), N-(3(R)-tetrahydrofuranyl)-6-aminopurine riboside (CVT-510,Tecadeonson), N6-cyclohexyl-2-O-methyladenosine (SDZ WAG 994), and N6-Cyclopentyl-N5′-ethyladenosine-5′-uronamide (Selodenoson).

Preferably the selective A₁ adenosine receptor agonist is ADAC.

Alternatively the selective A₁ adenosine receptor agonist is CCPA.

Alternatively the A₁ adenosine receptor agonist is a non-selective A₁ adenosine receptor agonist.

Preferably the non-selective A₁ adenosine receptor agonist is adenosine.

Preferably the A₁ adenosine receptor agonist is administered systemically.

Alternatively the A₁ adenosine receptor agonist is administered topically onto the round window membrane of the cochlea.

Preferably the A₁ adenosine receptor agonist is administered to a patient who has been exposed to acute or impulse noise.

Alternatively the A₁ adenosine receptor agonist is administered to a patient who has been exposed to prolonged excessive noise.

Preferably the A₁ adenosine receptor agonist is administered within about 24 hours of exposure to excessive noise.

More preferably the A₁ adenosine receptor agonist is administered within about 6 hours of exposure to excessive noise.

Preferably the A₁ adenosine receptor agonist is administered according to a dosage regime including more than one administration of the A₁ adenosine receptor agonist after exposure to excessive noise.

Preferably the A₁ adenosine receptor agonist is administered according to a dosage regime wherein the first administration is administered within about 24 hours of exposure to excessive noise.

More preferably the A₁ adenosine receptor agonist is administered according to a dosage regime wherein the first administration is administered within about 6 hours of exposure to excessive noise.

Preferably the A₁ adenosine receptor agonist is administered according to a dosage regime wherein the first administration is administered within about 6 hours of exposure to excessive noise and the remaining administrations are administered as single administrations at 24 hour intervals from the time of the first administration.

Preferably the A₁ adenosine receptor agonist is administered according to a dosage regime wherein the dosage regime includes at least 5 administrations of the A₁ adenosine receptor agonist.

Preferably the exposure to excessive noise does not exceed a noise level noise of 110 dB sound pressure level for 24 hours.

The invention in a third aspect provides the use of an A₁ adenosine receptor agonist in the manufacture of a medicament for the treatment of noise-induced hearing loss.

The invention in a fourth aspect provides the use of an A₁ adenosine receptor agonist in the manufacture of a medicament to reduce free radical damage in the cochlea after noise exposure.

Preferably the A₁ adenosine receptor agonist is a selective A₁ adenosine receptor agonist.

Preferably the selective A₁ adenosine receptor agonist is selected from the group including N6-cyclopentyl adenosine (CPA), 2-Chloro-N⁶-cyclopentyl adenosine (CCPA), S—N⁶-(2-endo-norbornyl)adenosine [S(−)-ENBA], adenosine amine congener (ADAC), ([1S-[1a,2b,3b,4a(S*)]]-4-[7-[[2-(3-chloro-2-thienyl)-1-methylpropyl]amino]-3H-imidazo-[4,5-b]pyridyl-3-yl]cyclopentane carboxamide) (AMP579), N—[R-(2-Benzothiazolyl)thio-2-propyl]-2-chloroadenosine (NNC-21-0136), N-[(1S, trans)-2-hydroxycyclopentyl]adenosine (GR79236), N-(3(R)-tetrahydrofuranyl)-6-aminopurine riboside (CVT-510,Tecadeonson), N6-cyclohexyl-2-O-methyladenosine (SDZ WAG 994), and N6-Cyclopentyl-N5′-ethyladenosine-5′-uronamide (Selodenoson).

Preferably the selective A₁ adenosine receptor agonist is ADAC.

Alternatively the selective A₁ adenosine receptor agonist is CCPA.

Alternatively the A₁ adenosine receptor agonist is a non-selective A₁ adenosine receptor agonist.

Preferably the non-selective A₁ adenosine receptor agonist is adenosine.

Preferably the medicament is formulated for administration to a patient who has been exposed to acute or impulse noise.

Alternatively the medicament is formulated for administration to a patient who has been exposed to prolonged excessive noise.

Preferably the medicament is formulated for administration within about 24 hours of exposure to excessive noise.

More preferably the medicament is formulated for administration within about 6 hours of exposure to excessive noise.

Preferably the medicament is formulated for administration according to a dosage regime including more than one administration of the A₁ adenosine receptor agonist.

Preferably the medicament is formulated for administration according to a dosage regime wherein the first administration is administered within about 24 hours of exposure to excessive noise.

Preferably the medicament is formulated for administration according to a dosage regime wherein the first administration is administered within about 6 hours of exposure to excessive noise.

Preferably the medicament is formulated for administration according to a dosage regime wherein the first administration is administered within about 6 hours of exposure to excessive noise and the remaining administrations are administered as single administrations at 24 hour intervals from the time of the first administration.

Preferably the medicament is formulated for administration according to a dosage regime wherein the dosage regime includes at least 5 administrations of the A₁ adenosine receptor agonist.

Preferably the exposure to excessive noise does not exceed a noise level noise of 110 dB sound pressure level for 24 hours.

Preferably the medicament is manufactured to be administered systemically.

Alternatively the medicament is manufactured to be administered topically onto the round window membrane of the cochlea.

Preferably the medicament reduces glutamate excitotoxicity in the cochlea after noise exposure.

Preferably the medicament increases blood flow and oxygen supply to the cochlea.

The invention in a fifth aspect provides the use of ADAC, including tautomeric forms, stereoisomers, polymorphs, pharmaceutically acceptable salts, and/or pharmaceutically acceptable solvates and/or chemical variants of ADAC, in the manufacture of a medicament for the treatment of noise-induced hearing loss.

The invention in a sixth aspect provides the use of ADAC, including tautomeric forms, stereoisomers, polymorphs, pharmaceutically acceptable salts, and/or pharmaceutically acceptable solvates and/or chemical variants of ADAC, in the manufacture of a medicament to reduce free radical damage in the cochlea after noise exposure.

The invention in a seventh aspect provides a method of treating noise-induced hearing loss in a mammal including the step of administering ADAC, including tautomeric forms, stereoisomers, polymorphs, pharmaceutically acceptable salts, and/or pharmaceutically acceptable solvates and/or chemical variants of ADAC, to the mammal.

The invention in an eighth aspect provides a method of treating tissue injury to the cochlea in a mammal after noise exposure including the step of administering ADAC, including tautomeric forms, stereoisomers, polymorphs, pharmaceutically acceptable salts, and/or pharmaceutically acceptable solvates and/or chemical variants of ADAC, to the mammal.

Further aspects of the present invention will become apparent from the following Figures and Examples, which are given by way of example only:

### BRIEF DESCRIPTION OF THE FIGURES

FIG. 1: shows auditory brainstem responses (ABRs) in rats exposed to 8-12 kHz band noise for 24 hours at 110 dB SPL. ABRs were measured in response to pure tones (4-24 kHz) and auditory clicks. ADAC (100 μg/kg i.p.) was administered as a single injection 6 hours or 24 hours after noise exposure, or as five injections administered every 24 hours commencing 6 hours post-noise (chronic treatment). In the control group, injections of the drug vehicle were administered at the same intervals as ADAC. Data are expressed as means±SEM. Animal numbers: n=8 per group. *p<0.05; **p<0.01; ***p<0.001; unpaired t-test.

FIG. 2: shows the threshold recovery (auditory brainstem responses, ABR) for rats treated with a single injection of ADAC or control solution 6 hours after noise exposure. (a) pure tones, (b) auditory clicks. *p<0.05; **p<0.01. Animal numbers: n=8 per group.

FIG. 3: shows the threshold recovery (ABR) in rats which received a single injection of ADAC or control solution 24 hours after noise exposure. (a) pure tones, (b) auditory clicks. *p<0.05; **p<0.01. Animal numbers: n=8 per group.

FIG. 4: shows (a) threshold recovery (ABR) in groups treated with 5 injections of ADAC or control solution. (a) pure tones, (b) auditory clicks. ***p<0.001. Animal numbers: n=8 per group.

FIG. 5: shows a comparison of different ADAC treatments on ABR threshold recovery. (a) pure tone audiogram, (b) auditory clicks. Animal numbers: n=8 per group.

FIG. 6: shows the rat organ of Corti (phalloidin staining) after treatment with (a) ADAC and (b) vehicle solution. Inner hair cells (IHC); Outer hair cells rows 1, 2, 3 (OHC1, OHC2, OHC3).

FIG. 7: shows nitrotyrosine immunostaining in the organ of Corti of (A) control and (B) ADAC-treated cochlea. Claudius cells (cc); inner hair cells (ihc); outer sulcus cells (osc); stria vascularis (sv); spiral ganglion neurones (sgn).

FIG. 8: shows body weight and temperature in animals treated with ADAC (100 μg/kg). A. Body weight was measured immediately before noise exposure and 14 days after noise exposure. B. Rectal temperature (° C.) was measured before ADAC administration and 30 and 60 minutes after the injection. Number of animals: n=8 per group.

FIG. 9: shows ABR threshold shifts in rats after exposure to 8-12 kHz band noise for 2 hours at 110 dB SPL (acute noise exposure). ABRs were measured in response to auditory clicks and pure tones (4-28 kHz) before and at time intervals (30 minutes and 14 days) after noise exposure. Five ADAC injections (100 μg/kg i.p.) were administered at 24 hour intervals commencing 6 hours post-noise. In the control group, injections of the vehicle solution were administered at the same intervals as ADAC. Data are expressed as means±SEM. Animal numbers: n=8 per group. *p<0.05; **p<0.01; unpaired t-test.

FIG. 10: shows the percentage hair cell loss in the cochlea exposed to noise for 2 hours. Data presented as means±SEM. Animal numbers: n=8 per group. *p<0.05, ***p<0.001; unpaired t-test.

FIG. 11: shows auditory brainstem responses (ABRs) in rats exposed to broad band noise for 24 hours at 110 dBSPL. ABRs were measured in response to auditory clicks (a) and pure tones (b-e) before noise exposure (baseline), 30 minutes after noise exposure (pre-treatment) and 48 hours after administration of adenosine receptor agonists (post-treatment). All drugs were delivered onto the cochlear round window membrane (f) Threshold recovery is defined as ABR post-treatment minus ABR pre-treatment. Data are expressed as means±SEM (n=8). *p<0.05; **p<0.01; ***p<0.001; one way ANOVA with Tukey's multiple comparison test. AP, artificial perilymph (control); adenosine (10 mM), non-selective adenosine receptor agonist; CCPA (1 mM), selective A₁ adenosine receptor agonist; CGS-21680 (0.2 mM), selective A_{2A} receptor agonist.

FIG. 12: shows the effect of adenosine receptor agonists and antagonists on summating potentials (SP) in rats kept at ambient noise levels (around 60 dB SPL). SP thresholds, representing the inner hair cell receptor potential, were measured at frequencies ranging from 4-26 kHz prior to perfusion of artificial perilymph (AP; baseline), after AP perfusion and after perfusion with adenosine receptor agonists adenosine and CCPA. Data presented as mean±SEM (n=8). *p<0.05 **p<0.01, one way ANOVA with Tukey's multiple comparison test. AP, artificial perilymph (control); adenosine (10 mM), non-selective adenosine receptor agonist; CCPA (1 mM), selective A₁ adenosine receptor agonist; CGS-21680 (0.2 mM), selective A_{2A} receptor agonist; SCH-58261, selective A_{2A} receptor antagonist.

FIG. 13: shows (A) nitrotyrosine immunostaining in the noise-exposed cochleae treated with adenosine receptor agonists (adenosine, CCPA) or vehicle solution (AP). No immunostaining was detected when the nitrotyrosine antibody was omitted. (B). Semi-quantitative analysis of nitrotyrosine immunoreactivity. Abbreviations: cc, Claudius cells; dc, Deiters' cells; hc, Hensen's cells; idc, interdental cells; is, inner sulcus cells; ihc, inner hair cells; ohc, outer hair cells; opc, outer pillar cells. Scale bars: 50 μm. Data are expressed as means±SEM (n=4 animals per group). **p<0.01; ***p<0.001; one way ANOVA with Tukey's multiple comparison test.

### DETAILED DESCRIPTION

The present invention relates generally to the use of A₁ adenosine receptor agonists in the treatment of hearing loss.

In a particularly preferred embodiment the invention relates to the use of A₁ adenosine receptor agonists in the manufacture of a medicament for the treatment of noise-induced hearing loss.

Adenosine receptors are present in most body tissues, including the cochlea of the inner ear. Adenosine has a role in tissue protection and recovery from stress. The inventors have found that the use of A₁ adenosine receptor agonists to treat noise-induced cochlear injury effectively recovers hearing sensitivity. It has previously been thought that A₁ adenosine receptor agonists only had a prophylactic use. As a result of that thinking, A₁ adenosine receptor agonists have been considered to have limited practical application.

In a preferred aspect, use of an A₁ adenosine receptor agonist can provide about 5-12 dB recovery of hearing after exposure to noise, or more preferably about 25-30 dB, or about 30-60%, of the hearing loss. From a practical perspective, in the clinic even a 5 dB improvement is significant. The improvements achieved by the present invention are therefore very significant.

Thus, the invention provides a method of treating noise-induced hearing loss, the method including the step of administering an A₁ adenosine receptor agonist.

A₁ adenosine receptor agonists can be either selective for A₁ receptors or broadly selective for all adenosine receptors (A₁, A_{2A}, A_{2B}, A₃). Thus A₁ adenosine receptor agonists as referred to throughout this specification, should be interpreted as including non-selective A₁ adenosine receptor agonists, such as adenosine, and selective A₁ adenosine receptor agonists, such as adenosine amine congener (ADAC) and 2-Chloro-N⁶-cyclopentyl adenosine (CCPA).

The A₁ adenosine receptor agonist according to a preferred embodiment of the invention will be a selective A₁ adenosine receptor agonist. Suitable selective A₁ adenosine receptors may be selected from the group including N6-cyclopentyl adenosine (CPA), 2-Chloro-N⁶-cyclopentyl adenosine (CCPA), S—N⁶-(2-endo-norbornyl)adenosine [S(−)-ENBA], adenosine amine congener (ADAC), ([1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide) (AMP579), N—[R-(2-Benzothiazolyl)thio-2-propyl]-2-chloroadenosine (NNC-21-0136), N-[(1S, trans)-2-hydroxycyclopentyl]adenosine (GR79236), N-(3(R)-tetrahydrofuranyl)-6-aminopurine riboside (CVT-510,Tecadeonson), N6-cyclohexyl-2-O-methyladenosine (SDZ WAG 994), and N6-Cyclopentyl-N5′-ethyladenosine-5′-uronamide (Selodenoson). In a particularly preferred embodiment the selective A₁ adenosine receptor agonist will be CCPA. In a more particularly preferred embodiment the selective A₁ adenosine receptor agonist will be ADAC.

According to an alternative embodiment of the invention, the A₁ adenosine receptor agonist may be a non-selective A₁ adenosine receptor agonist. A preferred non-selective A₁ adenosine receptor agonist for use in the present invention is adenosine. Where a non-selective A₁ adenosine receptor agonist is used in accordance with the present invention, a greater concentration will be required relative to the concentration of a selective A₁ adenosine receptor agonist.

Where an A₁ adenosine receptor agonist (e.g adenosine, ADAC or CCPA) is referred to throughout this specification, this should be interpreted as including the use of tautomeric forms, stereoisomers, polymorphs, pharmaceutically acceptable salts, pharmaceutically acceptable solvates, and/or chemical variants or the like, of the A₁ adenosine receptor agonist. As will be apparent to the skilled person, the various forms and/or variants referred to should not be of a type that would detrimentally affect the usefulness of the A₁ adenosine receptor agonist in this invention. A skilled person, once in possession of the invention disclosed herein would be well able to determine such matters.

The chemical structure of the selective A₁ adenosine receptor agonists, particularly ADAC, is extensively modified compared to adenosine, as shown below in Table 1.

TABLE 1
Adenosine and selective A₁ adenosine receptor agonists

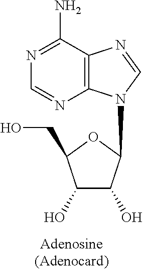

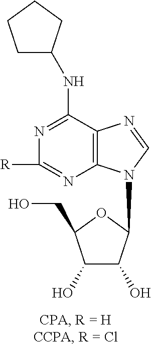

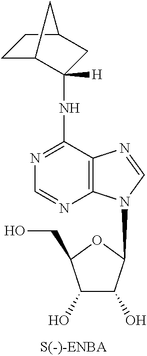

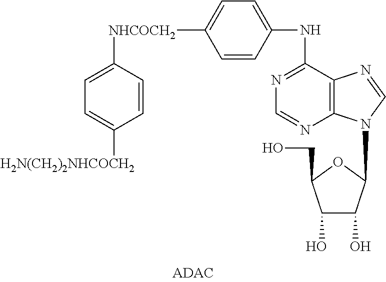

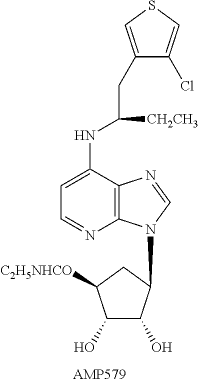

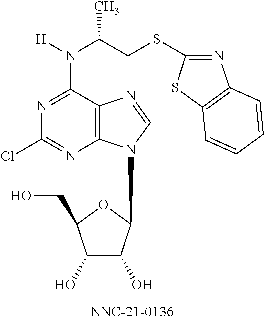

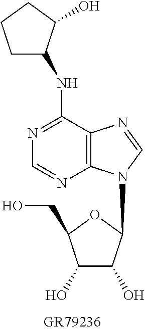

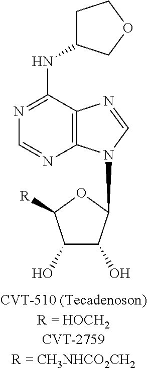

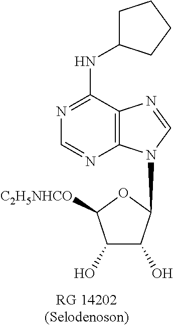

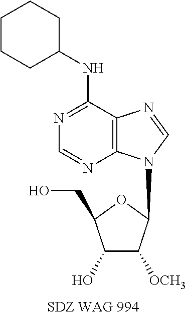

In one embodiment, the A₁ adenosine receptor agonist may be administered systemically thus avoiding the need to administer the treatment directly into the middle or inner ear (an office procedure required). The A₁ adenosine receptor agonist may be administered intraperitoneally, intravenously, orally, intramuscularly or subcutaneously to achieve this systemic effect. The most appropriate route for systemic delivery would at least in part depend on the pharmacological properties of the A₁ adenosine receptor agonist selected. Intraperitoneal administration is exemplified in the Experimental section.

Alternatively, if desired the A₁ adenosine receptor agonist may be formulated for topical administration to the inner ear by intratympanic injection, in particular onto the round window membrane of the cochlea. Intratympanic administration of a topical formulation is exemplified in the Experimental section. The advantage of this procedure is that any possible systemic side effect of the drug may be avoided.

Excessive noise is made up of two parts—the time of exposure and the loudness of the noise. Sustained exposure to noise above 85 decibels (dB) is considered to be excessive noise. The present invention can be used in connection with exposure to excessive noise over time, where that exposure is acute (for example, sustained excessive noise exposure for 2 hours) or prolonged (for example, sustained exposure for 24 hours), or where the exposure is to sudden loud noise (eg explosions or the like; known as impulse noise). Preferably the exposure to excessive noise does not exceed a noise level noise of 110 dB sound pressure level for 24 hours.

The A₁ adenosine receptor agonist should preferably be administered within about 24 hours of exposure to excessive noise. More preferably this should be within about 6 hours of exposure to excessive noise.

It is preferred that the A₁ adenosine receptor agonist is administered according to a dosage regime wherein the first administration is administered within about 6 hours of exposure to excessive noise and the remaining administrations are administered as single administrations every 24 hour from the time of the first administration.

It is further preferred that the A₁ adenosine receptor agonist is administered according to a dosage regime wherein the dosage regime includes at least 5 administrations of the A₁ adenosine receptor agonist.

ADAC has been used in the past to provide tissue protection in experimental models of cerebral ischemia and Huntington's disease [12-14]. It has been found to be particularly advantageous as a drug as it has reduced peripheral side effects [12] compared to other drugs that act upon adenosine A₁ receptors. Other drugs that act upon adenosine A₁ receptors may have cardiovascular side effects such as bradycardia and hypotension and hypothermia [15]. The lack of side effects caused by ADAC and its high affinity for A₁ receptors in the brain is believed to be at least partially due to its modified chemical structure and increased ability to cross the blood-brain or blood-perilymph barrier [16]. ADAC is therefore a particularly preferred A₁ receptor agonist for use in the present invention. The inventors have also found that adenosine and CCPA or other selective A₁ adenosine receptor agonists are suitable for topical administration onto the round window membrane by intratympanic injection (an office procedure). This avoids any risk of systemic side effects.

Formulations suitable for parenteral administration of A₁ adenosine receptor agonists, such as ADAC have been previously described [17]. These known formulations include aqueous and non-aqueous, isotonic sterile injection solutions and sterile suspensions that can include solubilisers, thickening agents, stabilisers and preservatives. The adenosine A₁ adenosine receptor agonists can be dissolved in saline, aqueous dextrose and related sugars solutions, an alcohol, such as ethanol, isopropanol, glycols etc. An example of ADAC formulation for parenteral administration is provided in the Methods and Materials of the Experimental section.

Formulations suitable for topical administration of A₁ adenosine receptor agonists also include aqueous and non-aqueous, isotonic sterile injection solutions and sterile suspensions that can include solubilisers, thickening agents, stabilisers and preservatives. The A₁ adenosine receptor agonists can be dissolved in saline, aqueous dextrose and related sugars solutions; an alcohol, such as ethanol, isopropanol, glycols etc. Examples of adenosine A₁ adenosine receptor agonist formulations for topical administration to the round window membrane are also provided in the Experimental section.

Medicaments currently in use in relation to the treatment of hearing loss, such as antioxidants are only useful prophylactically [8]. These known medicaments do little to aid recovery of hearing. The only means of recovering hearing currently available is a hearing aid. While hearing aids can intensify sound, they cannot completely recover speech discrimination. Hearing aids also have practical disadvantages to the user.

Exposure to excessive noise causes oxidative stress in the cochlea, leading to hearing loss. Oxidative stress in the cochlea continues up to 10 days after the cessation of noise exposure and determines the final level of tissue damage. The inventors believe that administration of an adenosine A₁ adenosine receptor agonist after noise exposure can increase the preservation of auditory function after noise exposure by increasing the production of antioxidants, countering toxic effects of free radicals and glutamate (reducing glutamate excitotoxicity in the cochlea after noise exposure), and improving cochlear blood flow and oxygen supply. This is likely to allow the adenosine A₁ adenosine receptor agonist to have a therapeutic effect on noise-induced hearing loss, recovering hearing thresholds and hence improve speech discrimination. Thus other aspects of the invention provide the use of adenosine A₁ adenosine receptor agonist to reduce free radical damage in the cochlea, and/or to treat tissue injury to the cochlea, after noise exposure thus treating noise-induced hearing loss in a patient in need thereof. The manufacture of suitable medicaments, and treatment regimes, has been discussed previously.

Experimental

In Experiments 1 and 2, Wistar rats were exposed to noise (8-12 kHz, 110 dB SPL for 2-24 hours). ADAC was then administered to the Wistar rats at 100 μg/kg/day. The ADAC was either administered as a single injection 6 hours after noise exposure, or as a single injection 24 hours after noise exposure, or as multiple injections, with the first injection of the multiple injections being administered 6 hours after noise exposure.

Hearing thresholds were assessed using auditory brainstem responses (ABRs) and the cellular damage was evaluated by quantitative histology (hair cell loss). ABR represents the activity of the auditory nerve and the central auditory pathways (brainstem/mid-brain regions) responding to the sound (clicks or pure tones). Nitrotyrosine marker was used for the immunohistochemical assessment of free radical damage.

The experimental work showed that ADAC dramatically improves ABR thresholds. Multiple injections of ADAC starting 6 hours after the cessation of noise exposure was found to be the most effective therapeutic regime. The ADAC treated cochleae demonstrated reduced hair loss and RNS immunoreactivity.

Experiment 1: The Effect of ADAC on Prolonged Noise Exposure (Systemic Delivery)

Materials and Methods

Animals

8-10 weeks old Male Wistar rats were used in this study.

Experimental Groups

TABLE 2
ADAC injection regime

| Group | Noise | | |
|---|---|---|---|
| number | exposure | Treatment | Treatment regime |
| Group 1 | 24 hours | ADAC | Single injection 6 hours PE |
| Group 2 | 24 hours | Vehicle (control) | Single injection 6 hours PE |
| Group 3 | 24 hours | ADAC | Single injection 24 hours PE |
| Group 4 | 24 hours | Vehicle (control) | Single injection 24 hours PE |
| Group 5 | 24 hours | ADAC | Multiple injections |
| Group 6 | 24 hours | Vehicle (control) | Multiple injections |

PE = post-exposure

Each ADAC group (n=8) had a corresponding control group, which was treated with the vehicle solution (n=8).

Adenosine Amine Congener

Adenosine amine congener (ADAC) was obtained from Dr Ken Jacobson (NIH, Bethesda, USA). ADAC (2.5 μg) was dissolved first in 100 μL of 1N HCl and then in 50 ml of 0.1 M PBS (pH 7.4), making a 50 μg/mL stock solution. This solution was aliquoted at 1 mL in eppendorf tubes, and stored at −20° C. for later use. When required, the ADAC aliquots were heated in a 37° C. water bath for 30 minutes before administration. ADAC injection dose was 100 pg/kg/day given intraperitoneally, 200 μl/100 g body weight.

Vehicle

The control vehicle solution was prepared by dissolving 100 μL of 1N HCL in 50 ml of 0.1 M PBS (pH 7.4), aliquoted in eppendorf tubes and also heated to 37° C. in a water bath for 30 minutes before injection. The same volume of vehicle solution (200 μl/100 g body weight intraperitoneally) was given to the control groups.

Noise Exposure

The rats were exposed to 8-12 kHz band noise presented for 24 hours at 110 dB SPL. This was done in a custom built acoustic chamber (Shelburg Acoustics, Sydney, Australia) with internal speakers and external controls (sound generator and frequency selector). Sound intensity inside the chamber was tested using a calibrated Rion NL-40 sound level meter to ensure minimal deviations of sound intensity (110±1 dB SPL). Up to 4 rats were placed in the chamber in a standard rat cage. They were introduced to the sound chamber at 1 hour intervals so that the timing of subsequent ABRs could be kept consistent for all rats.

Auditory Brainstem Responses

ABR represents the activity of the auditory nerve and the central auditory pathways (brainstem/mid-brain regions) responding to the sound (clicks or pure tones). ABRs were obtained by placing fine platinum electrodes subdermally at the mastoid region of the ear of interest (active electrode), scalp vertex (reference) and mastoid region of the opposite ear (ground electrode). A series of auditory clicks or pure tones (4-28 kHz) presented at varying intensity and thresholds generate electrical activity reflecting differing levels of auditory processing. The sound threshold of the ABR complex (waves I-IV) were determined by progressively attenuating the sound intensity until the waveform can no longer be observed.

The acoustic stimuli for ABR were produced and the responses recorded using a Tucker-Davis Technologies auditory physiology workstation (Alachua, Fla., USA).

All ABR measurements were performed in a sound attenuator chamber (Shelburg Acoustics, Sydney, Australia). Rats were anaesthetised with the mixture of Ketamine (75 mg/kg) and Xylazine (10 mg/kg) intraperitoneally, and then placed onto a heating pad, to maintain body temperature at 37° C. ABR potentials were evoked with digitally produced 5 ms tone pips (0.5 ms rise-fall time) at frequencies between 4 and 28 kHz in half-octave steps. Sound pressure level (SPL) was raised in 5 dB steps starting from 10 dB below threshold level to 90 dB SPL. Responses were averaged at each sound level (1024 repeats with stimulus polarity alternated), and response waveforms were discarded when peak-to-peak amplitude exceeded 15 μV. The ABR threshold was defined as the lowest intensity (to the nearest 5 dB) at which a response could be visually detected above the noise floor.

ABR thresholds were measured before and after noise exposure, and after ADAC/vehicle treatment. Post-noise ABR recordings were obtained 1 hour before the rats received their first ADAC or vehicle injection. This was 5 hours after noise exposure for groups 1, 2, 5 and 6 or 23 hours for groups 3 and 4 (Table 2). The final ABR measurements were obtained 18 hours after the last ADAC/vehicle injection.

Cochlear Extraction

After the last ABR measurement, rats were killed by Pentobarbitone overdose and cochleae removed for histological analysis. The isolated cochleae were kept in 4% Paraformaldehyde overnight, until further processing (decapsulation or decalcification).

Hair Cell Counts

After the overnight fixation, the cochlea was decapsulated in 0.1 M PBS, to isolate the organ of Corti. The organ of Corti was removed with fine forceps, and separated into the apical, middle and basal turns. Wholemount tissues of the organ of Corti were placed into a 24-well plate, and then permeabilised with 1% Trition-X in 0.1 M PBS for 1 hour. 1% Alexa Fluor 488 phalloidin (Invitrogen) dissolved in 0.1 M PBS was used to stain the hair cells and their stereocilia. Tissues were incubated in phalloidin for 40 minutes, washed with 0.1 M PBS 3×10 min and mounted onto glass slides using CitiFlour. The slides were visualised using a Zeiss epi-fluorescence microscope and processed with Axiovision v3.1 software, using dark field filter and 100×, 200×, and 400× magnification. Non-overlapping images were taken for the entire length of the cochlea, and the number of missing outer hair cells was counted for each turn and presented as a percentage of total number of hair cells.

Nitrotyrosine (NT) Immunohistochemistry

After overnight fixation in 4% PFA, rat cochleae were decalcified in a 5% EDTA solution for 7 days and cryoprotected in a 30% sucrose (in 0.1 M PB) solution overnight. The cochleae were snap-frozen in N-pentane, and stored at −80° C. until further processing. Frozen cochlear tissues were cryosectioned at 30 μm and transferred into 24-well plates (Nalge Nunc Int., Naperville, USA) containing the sterile 0.1M PBS, and permeabilised with 1% Triton X-100 for 1 hr. Non-specific binding sites were blocked with 10% normal goat serum (Vector Laboratories, Burlingame, Calif.). The nitrotyrosine antibody (BIOMOL Research Laboratories Inc., Plymouth, Pa., USA) was diluted 1:750 in 1.5% normal goat serum and 0.1% Triton X-100 in 0.1 M PBS. Tissue sections were incubated with the primary antibody overnight at 4° C. The primary antibody was omitted in control wells. The secondary antibody Alexa 488 goat anti-mouse IgG conjugate (Invitrogen) was diluted 1:400 in a 0.1 M PBS solution containing 1.5% normal goat serum and 0.1% Triton X-100. Tissue sections were incubated with the secondary antibody for 2 hours in the dark, then rinsed several times in PBS, mounted in fluorescence medium (DAKO Corporation, Carpinteria, Calif., USA) and screened for NT specific immunofluorescence using a confocal microscope (TCS SP2, Leica Leisertechnik GmbH, Heidelberg, Germany). Image acquisition was controlled by Scanware software (Leica). A series of 6-10 optical sections were collected for each specimen, and image analysis was performed on an optical section from the centre of the stack. The detection settings were not changed to allow comparison of relative staining densities between control and ADAC-treated cochleae.

Statistical Analysis

All data were entered into and analysed by Microsoft Excel and SPSS v.15. Results are presented as the mean±S.E.M. The comparison of ABR thresholds and hair cell loss was performed using a student's unpaired t-test assuming unequal variances. The a level was set at P=0.05.

Results

Auditory Thresholds after Extended (24 Hours) Noise Exposure

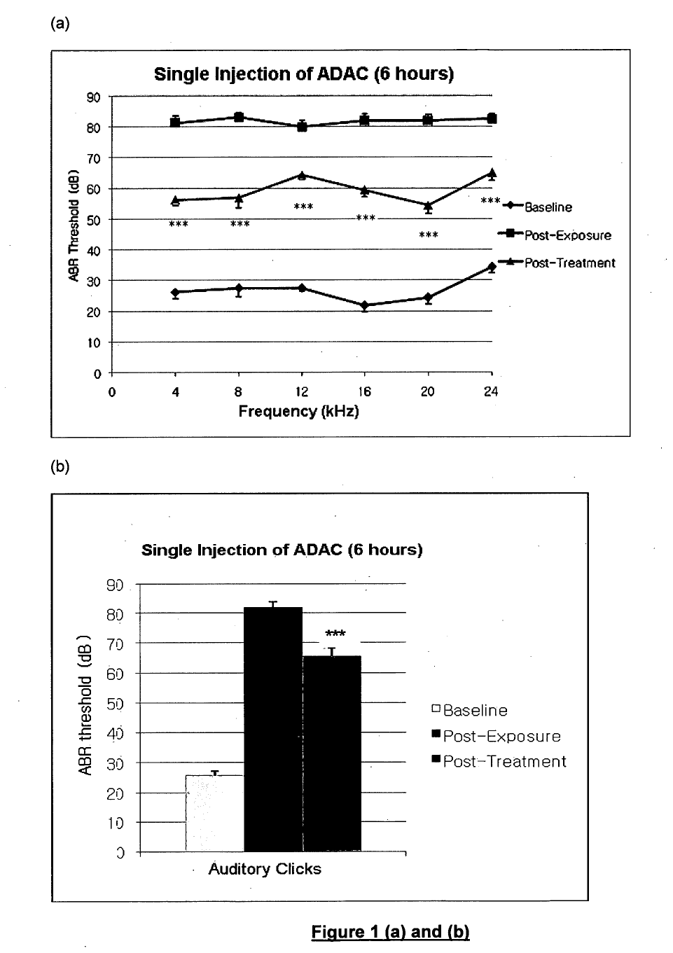

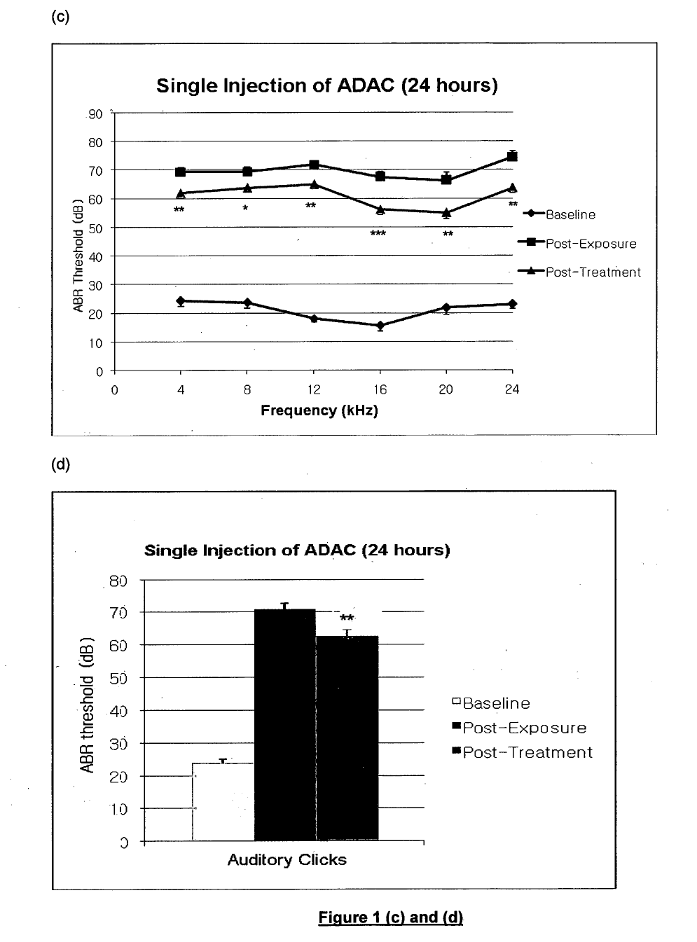

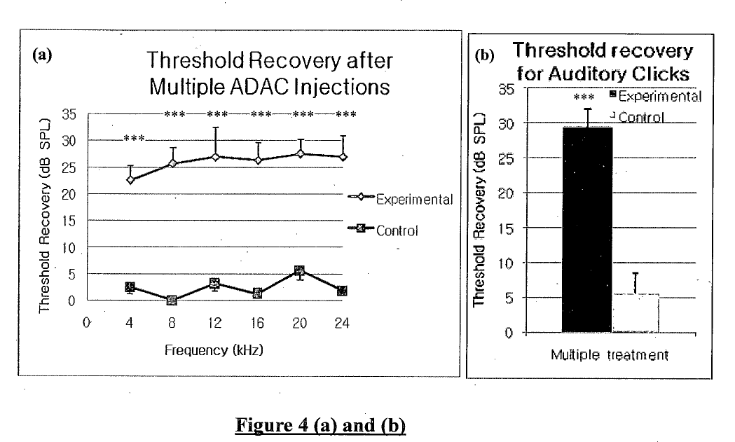

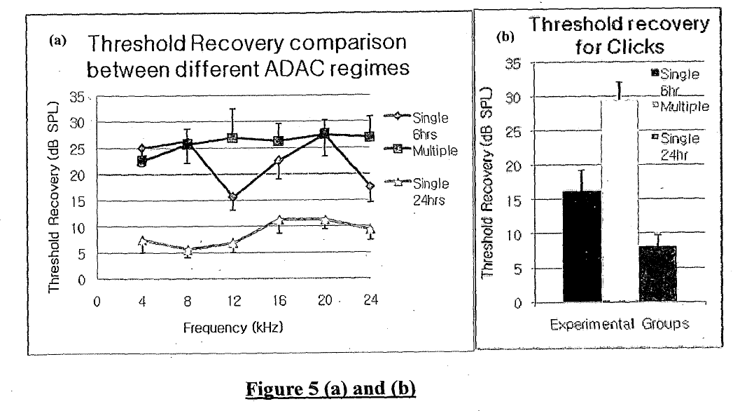

ABR thresholds were measured prior to noise exposure (baseline), post-exposure, and after ADAC treatment. Baseline ABR thresholds were comparable in all groups (FIG. 1). Threshold shifts within 24 hours after noise exposure ranged from 45 dB to 60 dB for auditory clicks and pure tones (FIG. 1). Animals treated with a single injection of ADAC showed substantial recovery of ABR thresholds: 17-26 dB when the animals received early treatment (6 hours after noise) and 5-12 dB in animals treated 24 hours after noise exposure. Chronic treatment with ADAC (5 days) provided uniform recovery of ABR thresholds at all pure tone frequencies (22-28 dB). Similar effect was observed for auditory clicks which have been plotted as separate bar graphs in FIG. 1. The highest recovery of ABR thresholds was observed in the group that received multiple injections of ADAC (29 dB±3 dB) (FIGS. 4 and 5) and the lowest in the group which received a single ADAC injection 24 hours after noise exposure (8±2 dB) (FIGS. 3 and 5). In control groups treated with the vehicle solution, ABR responses were not statistically different from post-exposure thresholds (FIG. 1).

Threshold Recovery

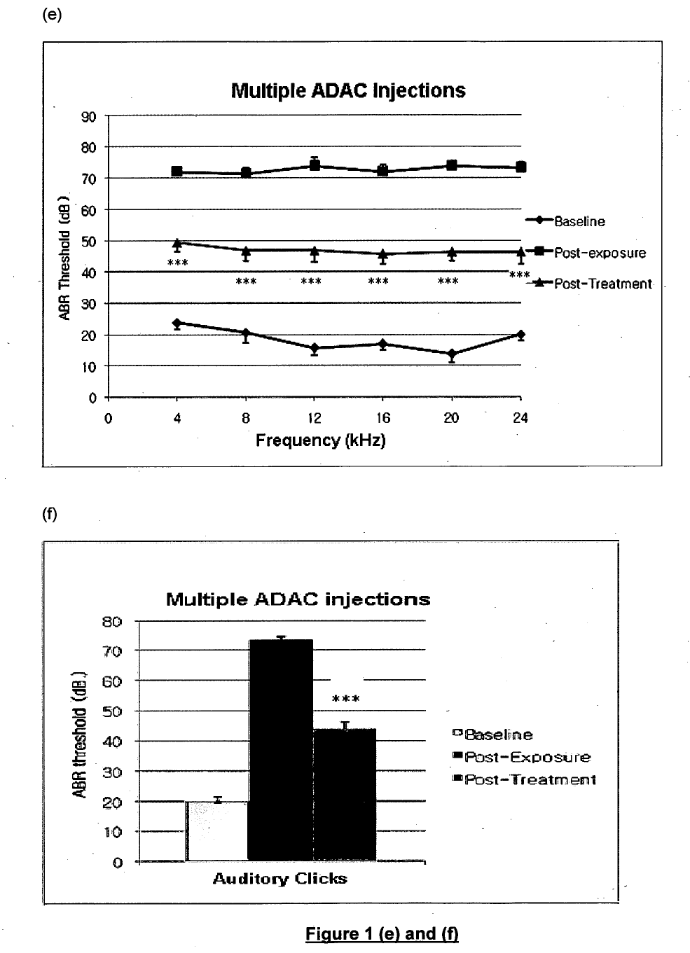

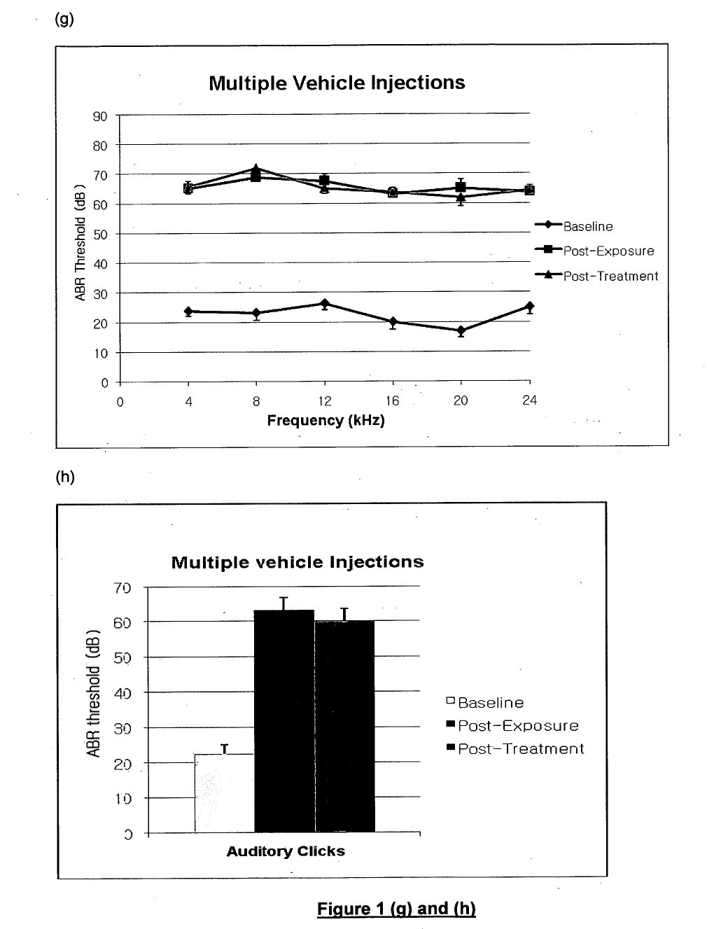

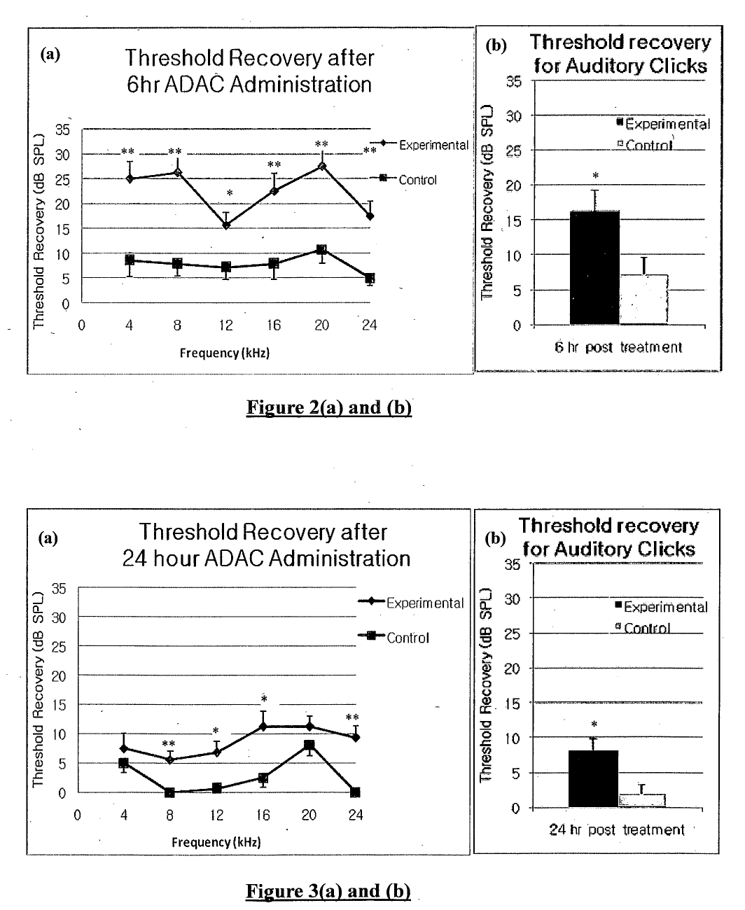

Threshold recovery is the difference between post-exposure and post-treatment thresholds. The comparison of ADAC-treated and control groups is shown in the FIGS. 2-5.

FIG. 2 demonstrates the threshold recovery in rats treated with a single injection of ADAC 6 hours after noise exposure. There was a statistically significant difference between the groups in the level of recovery (*p<0.05; **p<0.01) for pure tones and auditory clicks, however the level of recovery was not uniform across the frequencies tested, being the lowest at 12 and 24 kHz. A small recovery of hearing thresholds observed in control animals is due to temporary threshold shift (TTS).

FIG. 3 shows that the effect of ADAC administration on threshold recovery is less pronounced 24 hours after noise exposure.

As shown in FIG. 4, the best recovery of hearing thresholds (<25 dB) was observed with prolonged ADAC treatment (5 injections).

ADAC injections provide stable recovery in all frequencies, whereas a single ADAC injection is less effective at 12 kHz and 24 kHz pure tones, and auditory clicks. The late start of ADAC treatment (24 hours post-exposure) is the least effective treatment regime, as shown in FIG. 5.

Hair Cell Loss

Histological analysis of the organ of Corti exposed to noise (8-12 kHz, 110 dB SPL for 24 hours) demonstrated damages to the upper basal and the lower middle turn, whilst the apical turn was not affected. Representative examples of the basal turn organ of Corti are shown in FIG. 6. The organ of Corti in the control noise exposed cochlea treated with the vehicle solution showed a widespread outer hair cell loss particularly in the first row, and some inner hair cell loss (FIG. 6(a)). In contrast, the surface preparation of the organ of Corti from the ADAC-treated rat cochlea (FIG. 6(b)) showed well preserved hair cell morphology.

Nitrotyrosine (NT) Immunostaining

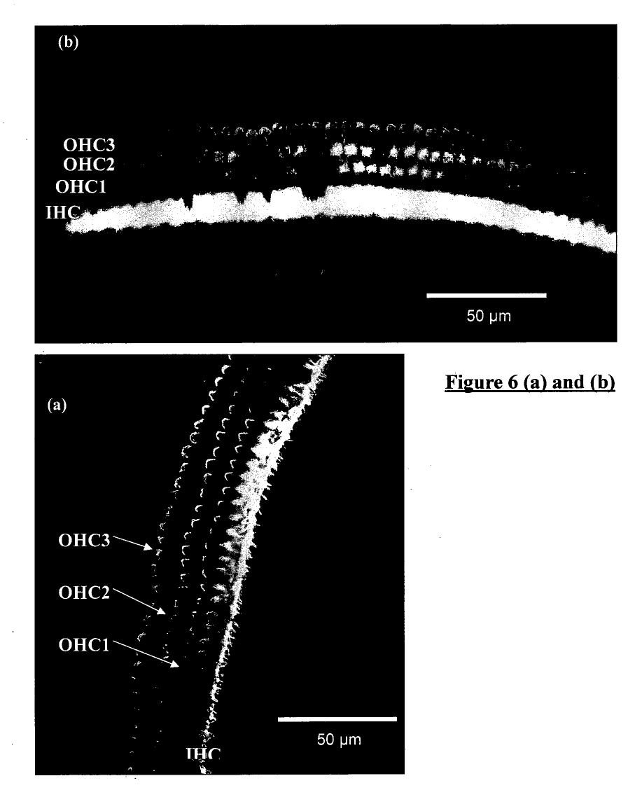

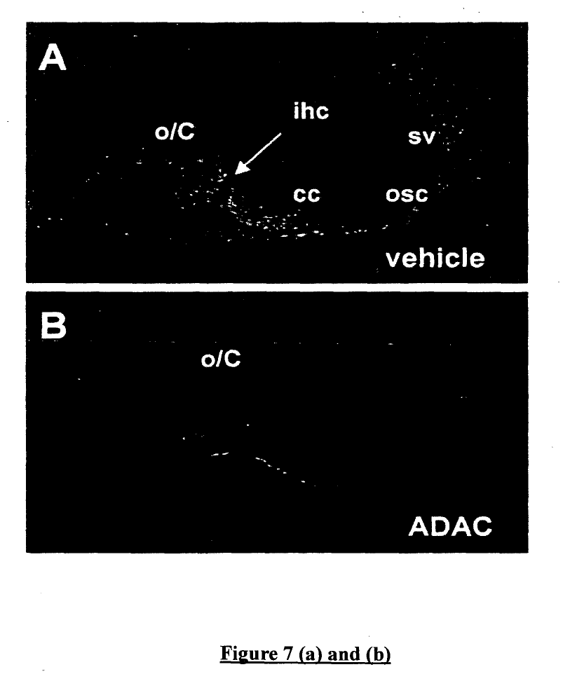

The vehicle treated rats showed NT immunoreactivity in the organ of Corti, and outer sulcus cells (FIG. 7A). In contrast, very little NT immunostaining was observed in corresponding tissues in the ADAC treated cochlea (FIG. 7B). Reduced NT immunoreactivity in ADAC-treated cochleae was indicative of low free radical activity.

Experiment 2: The Effect of ADAC on Acute Noise Exposure (Systemic Delivery)

Materials and Methods

Experimental Groups

TABLE 3
ADAC injection regime

| Group | Noise | | |
|---|---|---|---|
| number | exposure | Treatment | Treatment regime |
| Group 1 | 2 hours | ADAC | Multiple injections |
| Group 2 | 2 hours | Vehicle (control) | Multiple injections |

Animals

Male Wistar rats (8-10 weeks old) were used in this study.

Treatments

ADAC and aliquots and vehicle solutions were prepared as for Experiment 1.

Noise Exposure

Rats were exposed to 8-12 kHz band noise presented for 2 hours at 110 dB SPL. Noise exposures were carried out in a custom built acoustic chamber (Shelburg Acoustics,. Sydney, Australia) with internal speakers and external controls (sound generator and frequency selector). Sound intensity inside the chamber was tested using a calibrated Rion NL-40 sound level meter to ensure minimal deviations of sound intensity (110±1 dB SPL). Up to four rats were placed in the chamber in a standard rat cage.

Auditory Brainstem Responses

ABRs were obtained by placing fine platinum electrodes subdermally at the mastoid region of the ear of interest (active electrode), scalp vertex (reference) and mastoid region of the opposite ear (ground electrode). A series of auditory clicks or pure tones (4-28 kHz) presented at varying intensity and thresholds generate electrical activity reflecting differing levels of auditory processing. The sound threshold of the ABR complex (waves I-IV) were determined by progressively attenuating the sound intensity until the waveform can no longer be observed. The acoustic stimuli for ABR were produced and the responses recorded using a Tucker-Davis Technologies auditory physiology workstation (Alachua, Fla., USA).

All ABR measurements were performed in a sound attenuator chamber (Shelburg Acoustics, Sydney, Australia). Rats were anaesthetised with the mixture of Ketamine (75 mg/kg) and Xylazine (10 mg/kg) intraperitoneally, and then placed onto a heating pad, to maintain body temperature at 37° C. ABR potentials were evoked with digitally produced 5 ms tone pips (0.5 ms rise-fall time) at frequencies between 4 and 28 kHz in half-octave steps. Sound pressure level (SPL) was raised in 5 dB steps starting from 10 dB below threshold level to 90 dB SPL. Responses were averaged at each sound level (1024 repeats with stimulus polarity alternated), and response waveforms were discarded when peak-to-peak amplitude exceeded 15 μV. The ABR threshold was defined as the lowest intensity (to the nearest 5 dB) at which a response could be visually detected above the noise floor.

ADAC treatment commenced 6 hours after the cessation of noise exposure, whilst ABRs were recorded 30 minutes and 14 days after noise exposure.

Hair Cell Counts

The percentage of total number of hair cells was determined as for Experiment 1.

Statistical Analysis

The statistical analysis was carried out as for Experiment 1.

Results

Body Weight and Temperature

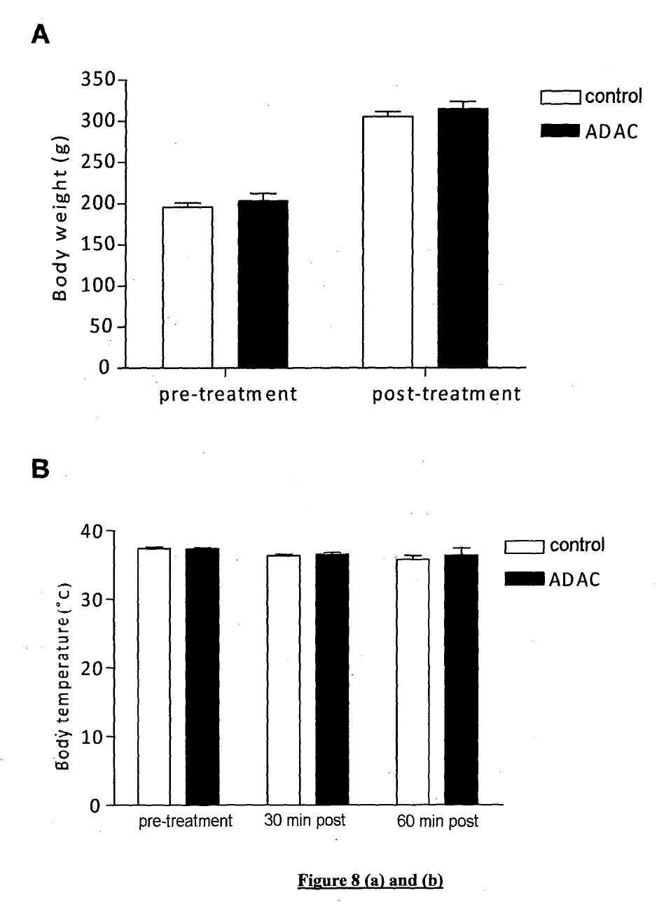

ADAC treatment did not induce overt behavioural changes in rats or alterations in body weight (FIG. 8(a)). In addition, body temperature remained stable after administration of ADAC (FIG. 8(b)).

Auditory Thresholds after Acute Noise Exposure

In this study, rats were exposed to 8-12 kHz band noise presented for 2 hours at 110 dB SPL. The same treatment regime was used as for Experiment 1: five ADAC injections given at 24 hour intervals. ABR recordings were made before and after noise exposure (30 min and 14 days).

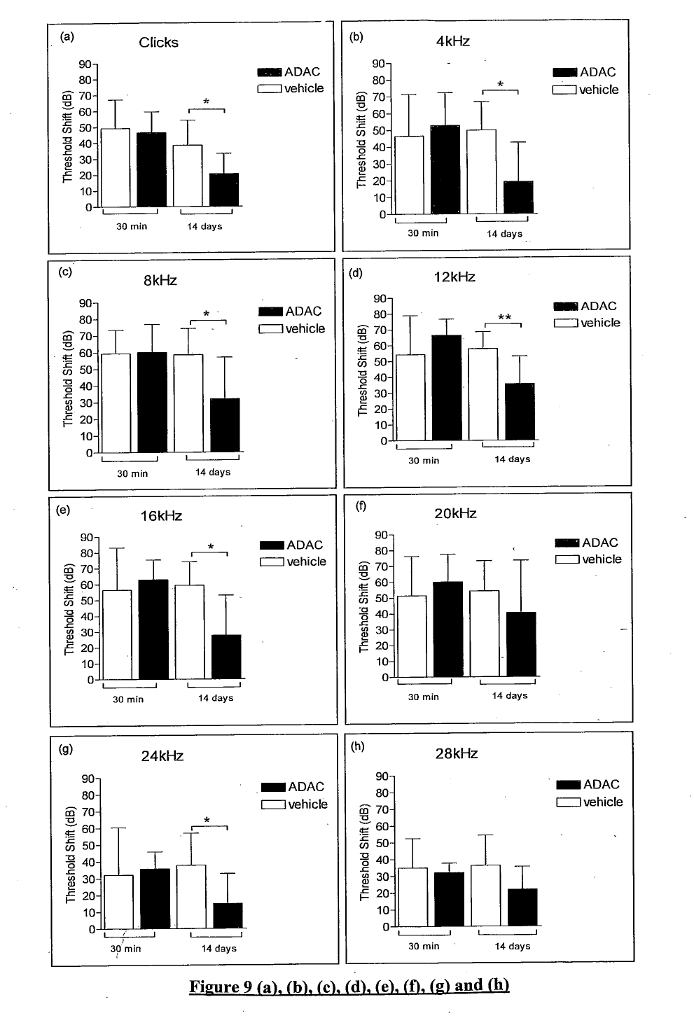

All noise exposed animals showed comparable threshold shifts (32-60 dB) for auditory clicks and pure tones (4-28 kHz) 30 minutes post-noise. The highest threshold shifts (55-60 dB) were observed at 8-12 kHz frequencies representing the most damaged area. At the end point of the study (14 days post-noise), threshold shifts were reduced in ADAC-treated animals compared to vehicle-treated controls (FIG. 9). Threshold recovery was the highest (up to 30 dB) at pure tone frequencies ranging from 4 to 16 kHz. ADAC effectively ameliorated hearing loss in rats exposed to acute noise.

Hair Cell Loss after Acute Noise Exposure

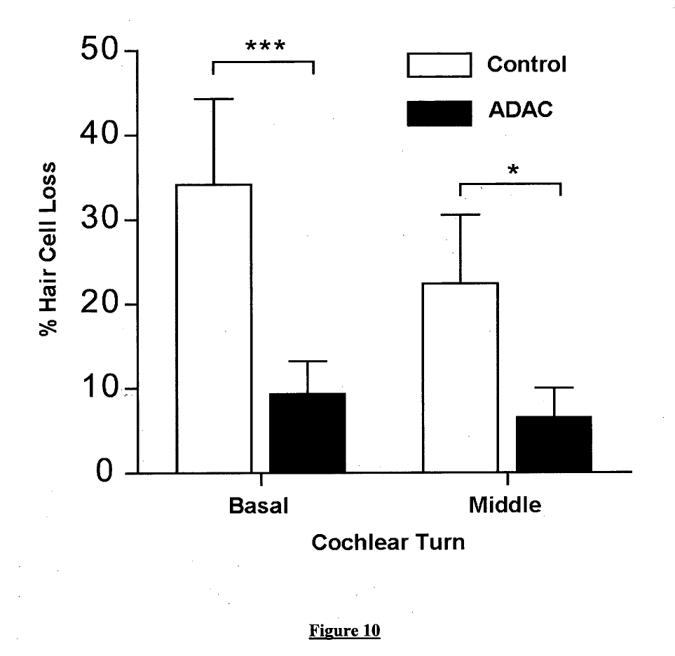

The outer and inner hair cells were counted in Alexa 488 phalloidin-labelled surface preparation of the organ of Corti in the basal, middle and apical turns and the percentage of missing hair cells was calculated for each turn. Quantitative analysis of the hair cell loss is shown in FIG. 10. The number of missing hair cells in control vehicle-treated animals varied between 23 and 34%, whilst the ADAC-treated animals showed on average 7-9% hair cell loss in the middle and basal cochlear turns respectively. Chronic ADAC treatment thus reliably reduced cellular lesion in the organ of Corti after traumatic noise exposure.

In the following experiment, selective adenosine receptor agonists were delivered onto the round window membrane (RWM) and compound action potentials (CAP), summating potentials (SP) or the auditory brainstem responses (ABR) were used to measure the effect of cochlear function before and after noise exposure.

Experiment 3—The Effect of Adenosine, CCPA and CGS-21680 on Acute Noise Exposure (Topical Delivery)

Materials and Methods

Drugs

The following adenosine receptor agonists and antagonists were purchased from Sigma-Aldrich: adenosine; CCPA (2-Chloro-N⁶-cyclopentyladenosine), an A₁ adenosine receptor agonist; CGS-21680 (2-p-(2-Carboxyethyl)phenethylamino-5′-N-ethylcarboxamidoadenosine hydrochloride hydrate), an A_{2A} receptor agonist; and SCH-58261 (7-(2-phenylethyl)-5-amino-2-(2-furyl)-pyrazolo-[4,3-e]-1,2,4-triazolo[1,5-c]pyrimidine), an A_{2A} receptor antagonist. Stock solutions of these compounds were prepared in artificial perilymph solution (AP; 122 mM NaCl, 18 mM NaHCO₃, 5 mM KCl, 0.7 mM CaCl₂, 0.5 mM MgCl₂, 4 mM D-glucose, 14 mM Mannitol in 5 mM HEPES, pH 7.5). Compounds were aliquoted and stored at −80° C.

Animals

The experiments were undertaken on male Wistar rats (8-10 weeks) with normal Preyer's reflex. Animals were supplied by the Vernon Jansen Unit (University of Auckland, New Zealand). All experimental procedures described in this study were approved by the University of Auckland Animal Ethics Committee.

Noise Exposure

Rats were exposed to a broadband noise presented for 24 hours at 90, 100, or 110 dBSPL. Noise exposures were carried out in a custom-built acoustic chamber (Shelburg Acoustics, Sydney, Australia) with internal speakers and external controls (sound generator and frequency selector). The sound levels in the cage were measured using a calibrated Rion NL-49 sound level meter to ensure minimal deviations of sound intensity. The animals had free access to food and water during the exposure.

Cochlear Perfusion with Adenosine Receptor Agonists and Assessment of Auditory Function

As a foundation for the noise studies and to determine the general effect of selective adenosine receptor agonists on the cochlea, auditory function was first evaluated in control animals using the summating potential (SP; measure of the inner hair cell receptor potential) and the compound action potential (CAP; measure of the neural afferent output). This was undertaken to determine the background influence of adenosine receptor activation in the normal cochlea as a platform for the studies in noise-exposed animals.

Animals were anaesthetized (sodium pentobarbital; 60 mg/kg i.p.) and placed on a thermostatically regulated blanket connected to a remote homeothermal control unit (Harvard Apparatus, Holliston, Mass., USA) to maintain stable body temperature (37.5° C.) via a rectal thermocouple probe (Harvard Apparatus). The head of the animal was placed on a heated (38° C. surface temperature) stereotaxic head-holder, connected to a heat block temperature controller (Bio-Medical Engineering Services, University of Auckland, New Zealand). The animals were artificially ventilated and the auditory bulla was exposed using a ventrolateral approach. The perfusion line was inserted close to the round window membrane (RWM). The RWM was perfused with test solutions containing A₁, or A_{2A} adenosine receptor agonists at 2.5 ml/min using a Harvard Apparatus Series PHD 22/2000 syringe pump. Adenosine receptor agonists adenosine (10 mM), CCPA (1 mM), CGS-21680 (200 μM), alone or in combination with adenosine receptor antagonist SCH-58261 (200 μM), were perfused for 90 minutes. Sound-evoked cochlear responses (CAP and SP) to pure tone stimuli (4-28 kHz) were recorded from a silver wire electrode placed onto the cochlear round window. These responses were measured using a Tucker-Davis System II for the presentation of tone stimuli and acquisition of the electrical potentials via a Grass P16 Pre-amplifier.

Auditory Brainstem Responses (ABR)

Auditory thresholds in noise-exposed animals were measured using auditory brainstem responses (ABR), which represent the sound evoked potentials from the auditory nerve and brainstem auditory nuclei. ABR measurements were recorded at least 24 hours prior to noise exposure (baseline) and then 30 min after noise exposure (pre-treatment). Adenosine receptor agonists or vehicle control were then delivered to the cochlear round window (around 6 hours post-noise) and ABR measurement was then repeated 48 hours after drug administration (post-treatment). ABR measurements were performed in a sound attenuator chamber (Shelburg Acoustics, Sydney, Australia). The rats were anesthetized with ketamine (75 mg/kg) and xylazine (10 mg/kg) and their body temperature was maintained at 38° C. with a heating pad as described. ABRs were obtained by placing fine platinum electrodes subdermally at the mastoid region of the ear of interest (active electrode), scalp vertex (reference) and mastoid region of the opposite ear (ground electrode). A series of auditory clicks or pure tones (4-28 kHz) presented at varying intensity and thresholds generated electrical activity reflecting differing levels of auditory processing. The sound threshold of the ABR complex (waves I-IV) were determined by progressively attenuating the sound intensity until the waveform was no longer observed. The acoustic stimuli for ABR were produced, and the responses, recorded, using a Tucker-Davis Technologies auditory physiology workstation (Alachua, Fla., USA) controlled by computer-based digital signal processing package and software (BioSig, Alachua, Fla., USA). ABR potentials were evoked with digitally produced 5 ms tone pips (0.5 ms rise-fall time) at frequencies between 4 and 28 kHz in half-octave steps. Sound pressure level (SPL) was raised in 5 dB steps starting from 10 dB below threshold level to 90 dB SPL. Responses were averaged at each sound level (1024 repeats with stimulus polarity alternated), and response waveforms were discarded when peak-to-peak amplitude exceeded 15 μV (artefact reject). The ABR threshold was defined as the lowest intensity (to the nearest 5 dB) at which a response could be visually detected above the noise floor. The animals were euthanised after hearing assessment and the cochleae collected for immunohistochemical assessment of free radical damage.

Administration of Adenosine Receptor Agonists into the Cochlea

Six hours after exposure to broad band noise (110 dBSPL for 24 hours), adenosine receptor agonists were delivered to the round window membrane (RWM) in the left cochlea, whilst the contralateral ear served as untreated control. The rats were anaesthetised with ketamine (75 mg/kg i.p.) and xylazine (10 mg/kg i.p.) and the auditory bulla opened by a dorsal approach to gain access to the middle ear and expose the cochlea under sterile conditions. Briefly, the incision was made medial and posterior to the pinna and the muscle was separated from the underlying bone of the auditory bulla. A small opening was made in the posterior region of the tympanic bulla using a scalpel blade to expose the RWM. The RWM was visualised under an operating microscope and a piece of gelatine sponge (Gelfoam; Upjohn, Kalamazoo, Mich.) soaked in 10 μL volume of test drug (adenosine, 10 mM; CCPA, 1 mM; CGS-21680, 200 μM) in saline was placed in the groove in direct contact with the RWM. In control experiments saline solution without test drug was applied onto the RWM. The bulla was then sealed with bone cement, the wound sutured and the animal allowed to recover. Auditory brainstem responses were measured 48 hours after surgery.

Assessment of Oxidative Stress by Nitrotyrosine Immunohistochemistry

Nitrotyrosine formation in the noise-exposed cochlea was assessed by immunohistochemistry. After overnight fixation in 4% PFA, noise-exposed and control rat cochleae were decalcified in a 5% EDTA solution for 7 days and cryoprotected in a 30% sucrose (in 0.1 M PB) solution overnight. The cochleae were then rinsed in 0.1 M phosphate buffer (PB), snap-frozen in isopentane at stored at −80° C. The cryosections (20 μm) were placed in 48-well plates (Nalge Nunc Int, Naperville, USA) containing sterile 0.1 M phosphate buffered saline (PBS, pH 7.4), permeabilised (1% Triton-X for 1 hour) and non-specific binding sites blocked (5% normal goat serum and 5% bovine serum albumin). Endogenous peroxidase activity was quenched by brief incubation with 0.3% H₂O₂. Sections were incubated overnight at 4° C. with a commercial antibody to nitrotyrosine (SA-468, BIOMOL, Plymouth Meeting, Pa., USA) at 1:500 dilution. In control reactions, the primary antibody was omitted. Immunoperoxidase reaction was detected using a secondary biotin-conjugated goat anti-rabbit IgG, followed by reaction visualisation using an avidin-biotin-peroxidase complex (ABC kit, Vector Laboratories) and diaminobenzidine (DAB kit, Vector). Immunostaining was observed using a microscope with Nomarski differential interference contrast optics (Zeiss Axioskop, Thornwood, N.Y., USA). Digital images were obtained with a digital camera (Zeiss Axiocam) and processed with AxioVision 4.7 software. Images were analyzed using identical acquisition parameters and immunolabeling was semi-quantified using ImageJ software (v.1.38x, NIH, USA). Images were deconvoluted (Colour Deconvolution 1.3 plugin) to differentiate DAB staining from the background and converted to 8-bit images. Regions of interest were selected and their immunostaining intensity histograms obtained and expressed as mean pixel intensity after greyscale conversion [23]. Between 15 and 32 images of the middle cochlear turn were analyzed in each group (n=4 animals per group) in a double-blind manner.

Statistical Analysis

Results are presented as the mean±S.E.M. Statistical analysis (comparison of hearing thresholds across frequency and treatment) was performed using a one-way ANOVA and Tukey's multiple comparison test. The a level was set at P=0.05.

Results

Adenosine and the Selective A₁ Adenosine Receptor Agonist CCPA Confer Protection to the Cochlea Following Noise Exposure

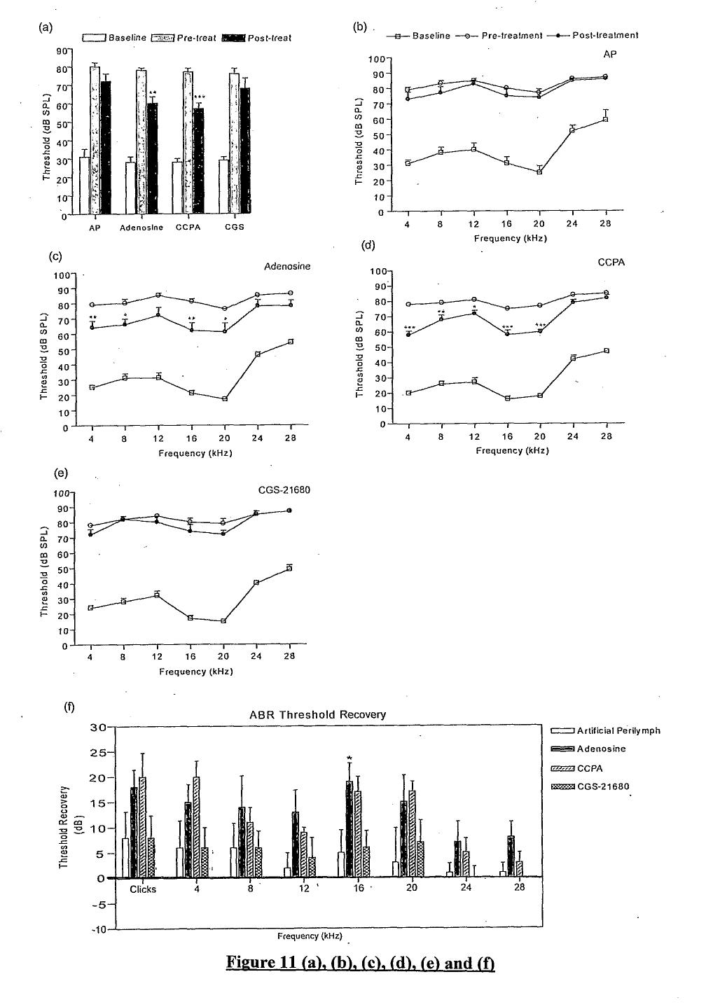

In this section of the experiment, rats were exposed to a broad-band noise for 24 hours at 110 dBSPL, and treated with a single dose of adenosine receptor agonist applied onto the RWM six hours after noise exposure. Functional assessment of hearing thresholds was performed 48 hours after treatment using auditory brainstem responses (ABR) to auditory clicks and pure tones (FIG. 11). ABR thresholds elevations from baseline following noise exposure (pre-treatment) were similar in all tested animals. Forty eight hours following adenosine and the selective A₁ adenosine receptor agonist CCPA administration to the RWM (post-treatment), animals showed markedly improved ABR thresholds for clicks and pure tones (FIGS. 11(a), (c) and (d)). In contrast, post-treatment thresholds remained unchanged in cochleae treated with CGS-21680 or control artificial perilymph (AP) solution (FIGS. 11(b) and (e)). Threshold recovery in different groups is presented in FIG. 11(f). Adenosine treated animals showed a threshold recovery of 18 dB for clicks and up to 19 dB for pure tones (16 kHz; p<0.01, one-way ANOVA). CCPA treated animals showed ABR threshold recovery of 20 dB for clicks and up to 20 dB for pure tones (FIG. 11(f)). There was a small amount of threshold recovery (1-7 dB) in control animals treated with the vehicle solution. Administration of selective A_{2A} receptor agonists CGS-21680 did not affect threshold recovery (FIG. 11(f)).

Baseline Measurements of Auditory Thresholds with Adenosine Receptor Agonists

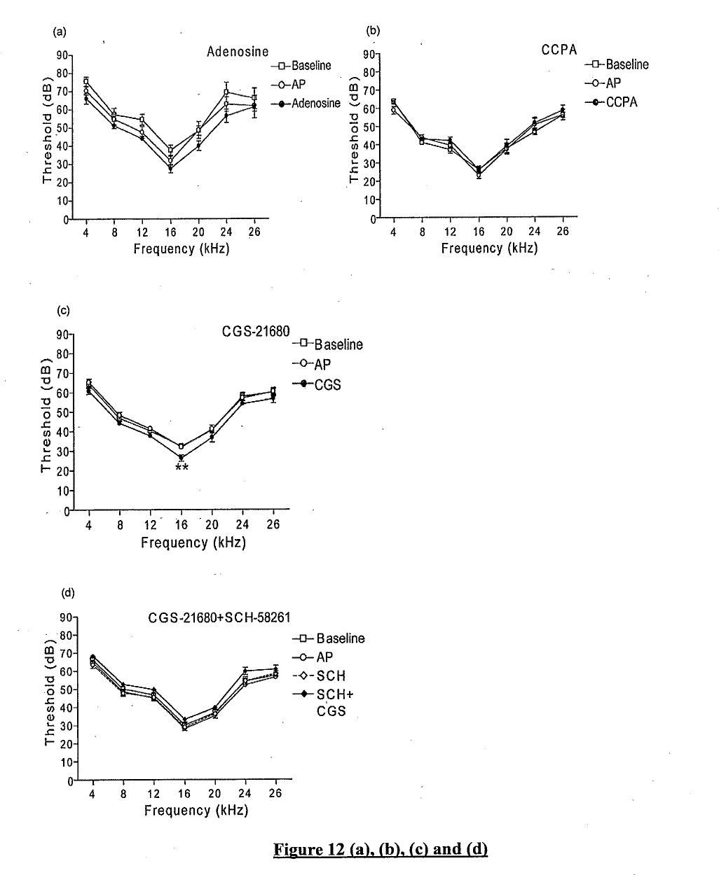

In control studies, the general effect of the various selective adenosine receptor agonists on baseline cochlear function were evaluated by electrocochleography, measuring summating potentials (SP) and compound action potentials (CAP) thresholds prior to cochlear perfusion (baseline), following control AP perfusion and after adenosine receptor agonist perfusions. Thresholds at baseline and after AP perfusion were comparable in each set of experiments (FIG. 12). Adenosine (10 mM) and the selective A₁ adenosine receptor agonist CCPA (1 mM) did not affect SP thresholds (FIGS. 12(a) and (b)), whilst the selective A_{2A} agonist CGS-21680 reduced SP thresholds by 5 dB at 16 kHz (FIG. 12(c)) (p<0.01, one-way ANOVA with Tukey's multiple comparison test). This reduction was inhibited by the A_{2A} receptor antagonist SCH-58261 (FIG. 12(d)). CAP thresholds were not altered by adenosine or any of selective adenosine receptor agonists (data not shown). Overall, there was a very limited influence of the selective adenosine receptor agonists on the cochlea at the hair cell or neural level.

Nitrotyrosine Immunoreactivity in the Noise-Exposed Cochleae

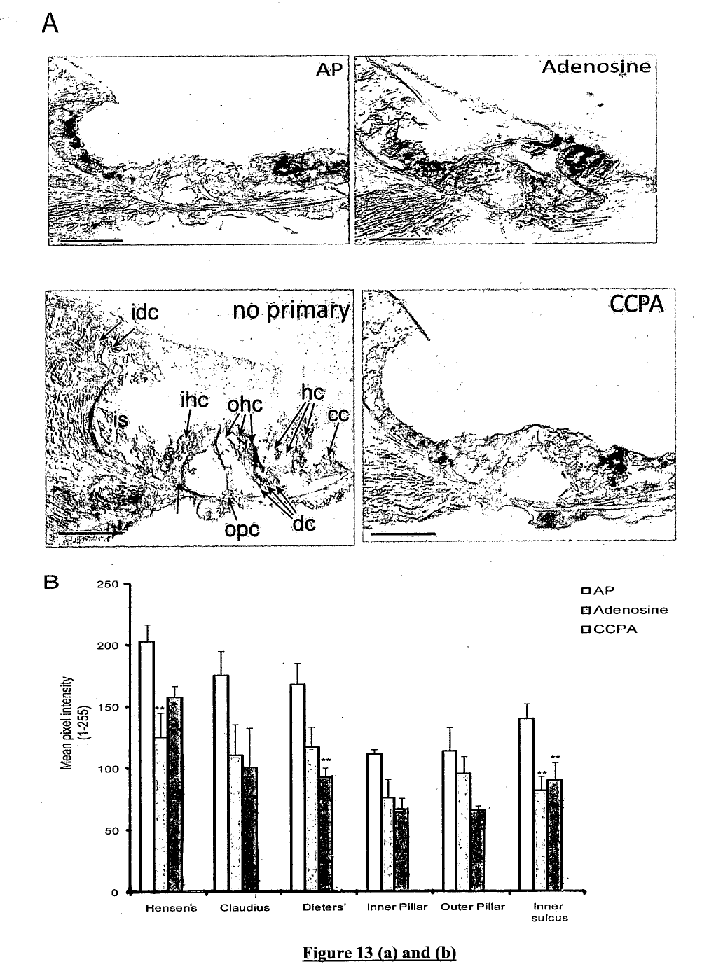

Nitrotyrosine formation in the noise-exposed cochlea was used as a marker of tissue damage from reactive nitrogen/oxygen species. The strongest nitrotyrosine immunostaining was found in the inner sulcus cells and supporting Hensen's cells (FIG. 13A). Nitrotyrosine immunoreactivity was also observed in other epithelial cells lining scala media (supporting Claudius, Dieters' and pillar cells in the organ of Corti). Very little staining in the sensory hair cells was observed. The spiral ligament, stria vascularis and the spiral ganglion neurones were unstained (data not shown). There was no immunolabelling in the non-noise exposed cochleae and when the primary antibody was omitted (FIG. 13A).

The distribution of nitrotyrosine immunostaining was similar in all noise-exposed cochleae. The intensity of immunolabelling was generally lower in the cochleae treated with adenosine or CCPA (FIGS. 13A,B) compared to vehicle-treated controls. In the adenosine treated cochleae, mean pixel intensity was reduced by 30-42% compared to AP control, particularly in the Hensen's and inner sulcus cells (p<0.01, one-way ANOVA). Similarly, the intensity of nitrotyrosine immunostaining was reduced by 22-45% in the CCPA treated cochleae, particularly in Dieters' and inner sulcus cells (p<0.01, one-way ANOVA

Conclusion

These examples show that stimulation of A₁ adenosine receptors mitigates noise-induced cochlear injury.

Treatment with A₁ adenosine receptor agonist after noise exposure leads to significant recovery of hearing thresholds. Earlier treatment starting at 6 hours after noise exposure provides greater recovery than late treatment starting at 24 hours after noise exposure. Prolonged treatment (5 injections) provides the best recovery of hearing thresholds and is recommended as a therapeutic approach in a clinical setting.

These examples also show that administration of an A₁ adenosine receptor agonist systemically, such as ADAC in Experiments 1 and 2, leads to significant recovery of hearing thresholds. Further, these examples show that administration of A₁ adenosine receptor agonists (e.g. adenosine (non-selective adenosine receptor agonist) and CCPA (selective A₁ adenosine receptor agonist)) topically onto the round window membrane improves auditory thresholds and reduces cellular injury in the organ of Corti.

The survival of sensory hair cells is increased by administration of A₁ adenosine receptor agonist, ADAC. Reduced hair cell loss and nitrotyrosine activity in the cochlea strongly support the cytoprotective and anti-oxidative role of the A₁ adenosine receptor agonist after noise-induced cochlear injury.

Nytrotyrosine immunochemistry (NT) was used for analysis of oxidative stress in the cochlea. NT is frequently used as a marker of free radical damage in the cochlea [20,21]. The overall intensity of NT immunostaining was reduced in the ADAC treated cochlea to a background level, suggesting strong anti-oxidant activity of ADAC. Adenosine applied onto the RWM also reduced the intensity of NT immunostaining.

No signs of systemic toxicity, such as the loss of body weight or changes in feeding or drinking behaviour or hypothermia have been observed with ADAC treatment.

Previous studies have demonstrated that drugs acting on adenosine receptors are useful prophylactically as they can prevent cochlear injury induced by noise or ototoxic drugs. The experimental results of this study show that adenosine receptor agonists have therapeutic effect in noise-induced hearing loss. A₁ receptors are strategically localised in the inner hair cells and the spiral ganglion neurons, and survival of these cells is crucial to cochlear recovery from noise stress.

The experimental evidence presented suggests that the activation of A₁ adenosine receptors reduces damage to the sensorineural tissues in the cochlea, leading to the functional recovery of hearing thresholds. The experimental evidence presented also suggests that administration may be systemic or topical.

These experimental examples strongly suggest that A₁ adenosine receptor agonists such as adenosine, ADAC and CCPA would be a valuable pharmacological treatment for noise-induced inner ear injury in humans, at least at sound pressure levels that do not exceed 110 dB for 2-24 hours. On the basis of the experimental examples, the inventors also believe that A₁ adenosine receptor agonists may be used in instances of exposure to acute or impulse noise and in instances of exposure to prolonged excessive noise. The treatment should be started as soon as possible after acoustic trauma, and the therapy should be continued for at least 5 days using one of the preferred routes of administration. The benefits to a patient requiring treatment for noise-induced hearing loss are important. That these treatment benefits can be provided to such a patient by use of an A₁ adenosine receptor agonist is surprising given the importance of those benefits.

The foregoing describes the invention including a preferred form thereof. Alterations and modifications as would be readily apparent to a person skilled in this art are intended to be included within the scope of the invention disclosed.

The reference to any prior art in this specification is not, and should not be taken as, an acknowledgment or any form of suggestion that that prior art forms part of the common general knowledge in any particular country.

### REFERENCES

1. Corwin J T (1998) Identifying the genes of hearing, deafness and disequilibrium. Proc. Natl. Acad. Sci. 95, 12080-12082
2. Kopke R, Allen K A, Henderson D, Hoffer M, Frenz D, Van de Water T. (1999) A radical demise. Toxins and trauma share common pathways in hair cell death. Ann. N. Y. Acad. Sci. 884:171-191.
3. Henderson D, Bielefeld E C, Harris K C, Hu B H. (2006) The role of oxidative stress in noise-induced hearing loss. Ear Hear. 27(1):1-19.
4. Vlajkovic S M, Abi S, Wang C J, Housley G D, Thorne P R. (2007) Differential distribution of adenosine receptors in rat cochlea. Cell Tissue Res. 328(3):461-471.
5. Ramkumar V, Whitworth C A, Pingle S C, Hughes L F, Rybak L P. (2004) Noise induces A1 adenosine receptor expression in the chinchilla cochlea. Hear. Res. 188(1-2):47-56.
6. Hu B H, Zheng X Y, McFadden S L, Kopke R D, Henderson D. (1997) R-phenylisopropyladenosine attenuates noise-induced hearing loss in the chinchilla. Hear. Res. 113(1-2):198-206.
7. Hight N G, McFadden S L, Henderson D, Burkard R F, Nicotera T. (2003) Noise-induced hearing loss in chinchillas pre-treated with glutathione monoethylester and R-PIA. Hear. Res. 179(1-2):21-32
8. LePrell C G, Yamashita D, Minami S B, Yamasoba T, Miller J M. (2007) Mechanisms of noise-induced hearing loss indicate multiple methods of prevention. Hear. Res. 226:22-43.
9. U.S. Pat. No. 6,177,434: Prevention or reversal of sensorineural hearing loss (SNHL) through biologic mechanisms
10. Richardson R T, Noushi F, O'Leary S (2006) Inner ear therapy for neural preservation. Audiol. Neuro-Otol. 11:343-356.
11. Fredholm B B (2007) Adenosine, an endogenous distress signal, modulates tissue damage and repair. Cell Death Differ. 14(7):1315-1323.
12. Von Lubitz D K J E, Lin, R C-S, Paul I A, Beenhakker M, Boyd M, Bischofberger N, Jacobson K A (1996) Postischemic administration of adenosine amine congener (ADAC): analysis of recovery in gerbils. Eur. J. Pharmacol. 316:171-179.
13. Von Lubitz D K J E, Lin R C-S, Bischofberger N, Beenhakker M, Boyd M, Lipartowska R, Jacobson K A (1999) Protection against ischemic damage by adenosine amine congener, a potent and selective adenosine A1 receptor agonist. Eur. J. Pharmacol 369, 313-317.
14. Blum D, Gall D, Galas M-C, D'alcantara P, Bantunbungi K, Schiffmann S N. (2002) The adenosine A₁ receptor agonist adenosine amine congener exerts a neuroprotective effect against the development of striatal lesions and motor impairments in the 3-nitropropionic acid model of neurotoxicity. J. Neurosci., 22:9122-9133.
15. Jacobson K A, Gao Z G. (2006) Adenosine receptors as therapeutic targets. Nat Rev Drug Discov. 5(3):247-264.
16. Jacobson K A, Daly J W (1991) Purine functionalized congeners as molecular probes for adenosine receptors. Nucleosides & Nucleotides 10:1029-1038.
17. WO/1997/037667: Use of an A₁ adenosine receptor agonist to treat cerebral ischaemia.
18. Yamashita D, Jiang H Y, Schacht J, Miller J M (2004) Delayed production of free radicals following noise exposure. Brain Res. 1019(1-2):201-209.
19. Knutsen L J, Lau J, Petersen H, Thomsen C, Weis J U, Shalmi M, Judge M E, Hansen A J, Sheardown M J. (1999) N-substituted adenosines as novel neuroprotective A(1) agonists with diminished hypotensive effects. J Med Chem. 42(18):3463-77.
20. Yamashita D, Jiang H Y, Le Prell C. G., Schacht J, Miller J M (2005) Post-exposure treatment attenuates noise-induced hearing loss. Neurosci., 134(2), 663-642.
21. Jiang H, Talaska A. E., Schacht J, Sha S. H. (2007) Oxidative imbalance in the agin inner ear, Neurobiol. Aging, 28(1), 1605-1612.
22. Livak K J and Schmittgen T D (2001), Analysis of relative gene expression data using real-time quantitative PCR and the 2^{-ΔΔC}^{_{T}} method. Methods 25(4):402-408.
23. Vlajkovic S M, Housley G D, Munoz D J, Robson S C, Sevigny J, Wang C J and Thorne P R, 2004. Noise exposure induces up-regulation of ecto-nucloside triphosphate diphosphohydrolases 1 and 2 in rat cochlea, Neuroscience 126, 763.
24. Jacobsen K. A. and Zhan-Guo, Adenosine receptors as therapeutic targets, Nature, March 2006, 250

1. A method of treating noise-induced hearing loss after noise exposure, the method including the step of administering an A₁ adenosine receptor agonist.

2. A method of treating tissue injury to the cochlea after noise exposure, the method including the step of administering an A₁ adenosine receptor agonist.

3. A method according to claim 1 wherein the A₁ adenosine receptor agonist is a selective A₁ adenosine receptor agonist.

4. A method according to claim 3 wherein the selective A₁ adenosine receptor agonist is selected from the group including N6-cyclopentyl adenosine (CPA), 2-Chloro-N⁶-cyclopentyl adenosine (CCPA), S—N6-(2-endo-norbornyl)adenosine [S(−)-ENBA], adenosine amine congener (ADAC), ([1S-[1a,2b,3b,4a(S*)]]-4-[7-[[2-(3-chloro-2-thienyl)-1-methylpropyl]amino]-3H-imidazo[4,5-b]pyridyl-3-yl]cyclopentane carboxamide) (AMP 579), N—[R-(2-Benzothiazolyl)thio-2-propyl]-2-chloroadenosine (NNC-21-0136), N-[(1S, trans)-2-hydroxycyclopentyl]adenosine (GR79236), N-(3(R)-tetrahydrofuranyl)-6-aminopurine riboside (CVT-510,Tecadeonson), N6-cyclohexyl-2-O-methyladenosine (SDZ WAG 994), and N6-Cyclopentyl-N5′-ethyladenosine-5′-uronamide (Selodenoson).

5. A method according to claim 4 wherein the selective A₁ adenosine receptor agonist is ADAC.

6. A method according to claim 4 wherein the selective A₁ adenosine receptor agonist is CCPA.

7. A method according to claim 1 wherein the A1 adenosine receptor agonist is a non-selective A1 adenosine receptor agonist.

8. A method according to claim 7 wherein the non-selective A₁ adenosine receptor agonist is adenosine.

9. A method according to claim 1 wherein the A₁ adenosine receptor agonist is administered systemically.

10. A method according to claim 1 wherein the A₁ adenosine receptor agonist is administered topically onto the round window membrane of the cochlea.

11. A method according to claim 1 wherein the A₁ adenosine receptor agonist is administered to a patient who has been exposed to acute or impulse noise.

12. A method according to claim 1 wherein the A₁ adenosine receptor agonist is administered to a patient who has been exposed to prolonged excessive noise.

13. A method according to claim 1 wherein the A₁ adenosine receptor agonist is administered within about 24 hours of exposure to excessive noise.

14. A method according to claim 1 wherein the A₁ adenosine receptor agonist is administered within about 6 hours of exposure to excessive noise.

15. A method according to claim 1 wherein the A₁ adenosine receptor agonist is administered according to a dosage regime including more than one administration of the A1 adenosine receptor agonist after exposure to excessive noise.

16. A method according to claim 15 wherein the A₁ adenosine receptor agonist is administered according to a dosage regime wherein the first administration is administered within about 24 hours of exposure to excessive noise.

17. A method according to claim 15 wherein the A₁ adenosine receptor agonist is administered according to a dosage regime wherein the first administration is administered within about 6 hours of exposure to excessive noise.

18. A method according to claim 17 wherein the A₁ adenosine receptor agonist is administered according to a dosage regime wherein the first administration is administered within about 6 hours of exposure to excessive noise and the remaining administrations are administered as single administrations at 24 hour intervals from the time of the first administration.

19. A method according to 15 wherein the A₁ adenosine receptor agonist is administered according to a dosage regime wherein the dosage regime includes at least 5 administrations of the A₁ adenosine receptor agonist.

20. A method according to claim 1 wherein the exposure to excessive noise does not exceed a noise level noise of 110 dB sound pressure level for 24 hours.

21. (canceled)

22. The method according to claim 1 wherein the treatment reduces free radical damage in the cochlea after noise exposure.

23-40. (canceled)

41. The method according to claim 1 wherein the treatment reduces glutamate excitotoxicity in the cochlea after noise exposure.

42. The method according to claim 1 wherein the treatment increases blood flow and oxygen supply to the cochlea.

43-44. (canceled)

45. A method according to claim 1 wherein the A1 adenosine receptor agonist is administered to a mammal, and wherein the A₁ adenosine receptor agonist is ADAC, including tautomeric forms, stereoisomers, polymorphs, pharmaceutically acceptable salts, and/or pharmaceutically acceptable solvates and/or chemical variants of ADAC.

46. A method according to claim 2 wherein the A1 adenosine receptor agonist is administered to a mammal after noise exposure, and wherein the A1 adenosine receptor agonist is ADAC, including tautomeric forms, stereoisomers, polymorphs, pharmaceutically acceptable salts, and/or pharmaceutically acceptable solvates and/or chemical variants of ADAC.

47-50. (canceled)
